(19) United States
(12) Patent Application Publication
(10) Pub. No.: US 2017/0009263 A1
(43) Pub. Date: Jan. 12, 2017

(54) METHODS AND HOST CELLS FOR ENHANCING PRODUCTION OF 1, 3-BUTANEDIOL

(71) Applicant: INVISTA NORTH AMERICA S.A.R.L., Wilmington, DE (US)

(72) Inventors: Alex Van Eck CONRADIE, Eaglescliffe (GB); Mariusz Stanislaw KAMIONKA, Middlesbrough (GB)

(21) Appl. No.: 15/204,008

(22) Filed: Jul. 7, 2016

Related U.S. Application Data
(60) Provisional application No. 62/190,169, filed on Jul. 8, 2015.

(51) Int. Cl. C12P 7/18; C07C 31/20; C12N 9/10; C12N 9/88; C12N 9/02

(52) U.S. Cl. CPC C12P 7/18; C12N 9/88; C12N 9/0008; C12N 9/13; C12Y 401/01005; C12Y 102/0101; C12Y 208/03; C07C 31/207

(57) ABSTRACT

This application describes non-naturally occurring host cells for enhanced 1,3-butanediol (1,3-BDO) production, methods for producing 1,3-BDO using such non-naturally occurring host cells, and 1,3-BDO products produced by such non-naturally occurring host cells and methods.

### BACKGROUND

1,3-butanediol (1,3-BDO) is a four-carbon diol commonly used as an organic solvent for food flavoring agents. It is also used as a co-monomer for polymer resins, and is widely employed as a hypoglycaemic agent. Optically active 1,3-BDO is a useful starting material for the synthesis of biologically active compounds and liquid crystals. In addition, dehydration of 1,3-BDO affords 1,3-butadiene, a chemical used to manufacture synthetic rubbers (e.g. tires), latex, and resins.

Several pathways are known for producing 1,3-butanediol, including those disclosed in U.S. Pat. No. 8,268,607 and U.S. Pat. No. 9,179,893, which are herein incorporated by reference in their entireties. For example, 1,3-BDO can be produced through a series of enzymatic conversions, as set forth in FIG. 3, that involve: (1) an acetyl-CoA C-acetyltransferase to yield acetoacetyl-CoA; (2) an acetoacetyl-CoA reductase or a 3-hydroxybutyryl-CoA dehydrogenase to yield 3-hydroxybutyryl-CoA; (3) a CoA transferase to yield 3-hydroxybutyrate; (4) a carboxylate reductase to yield 3-hydroxybutanal; and (5) a dehydrogenase to yield 1,3-BDO.

### SUMMARY

When the 1,3-BDO pathway of FIG. 3 is performed in a cell that naturally expresses an acetaldehyde dehydrogenase enzyme or an acetolactate decarboxylase enzyme, carbon flux that could be used for the production of 1,3-BDO will be shunted towards the production of ethanol or 1,2-BDO, thus decreasing the efficiency of 1,3-BDO expression (see, for example, FIG. 1). The present inventors have recognized that it would be beneficial to develop host cells and methods for more efficient production of 1,3-BDO.

The inventors have determined that it is possible to reduce or prevent carbon flux to ethanol in the 1,3-BDO pathway set forth in FIG. 1 by attenuating the activity of an acetaldehyde dehydrogenase enzyme. Attenuating the activity of an acetaldehyde dehydrogenase enzyme reduces or prevents the conversion of acetyl-CoA to acetaldehyde, and thereby reduces or prevents the production of ethanol. As a result, the inventors have developed a way to save two reducing equivalents in the 1,3-BDO pathway set forth in FIG. 1.

The inventors have further determined that it is possible to reduce or prevent carbon flux to 2,3-butanediol in the 1,3-BDO pathway set forth in FIG. 1 by attenuating the activity of an acetolactate decarboxylase enzyme. Attenuating the activity of an acetolactate decarboxylase enzyme reduces or prevents the conversion of acetolactate to acetoin, and thereby reduces or prevents the production of 2,3-butanediol. As a result, the inventors have developed a way to save one reducing equivalent in the 1,3-BDO pathway set forth in FIG. 1.

The inventors have further determined that it is possible to reduce carbon flux to 2,3-butanediol by regulating CO₂ partial pressure during fermentation.

The inventors have further determined that by using a CoA transferase that accepts acetyl-CoA and 3-hydroxybutyryl-CoA, the acetate byproduct of acetyl-CoA phosphorylation and de-phosphorylation can be shunted back to acetyl-CoA to further improve efficiency of 1,3-BDO production.

Thus, in one embodiment, there is a non-naturally occurring 1,3-BDO pathway having modified NAD(P)H balancing. In one embodiment, the pathway gains three reducing equivalents, relative to a comparable unmodified process. In one embodiment, the pathway gains three chemical species capable of transferring the equivalent of one electron in a redox reaction, relative to a comparable unmodified process. In one embodiment, the pathway forms more 1,3-butanediol, relative to a comparable unmodified process.

In one embodiment, there are non-naturally occurring host cells capable of producing 1,3-butanediol via this non-naturally occurring 1,3-BDO pathway. In one embodiment the non-naturally occurring host cells are capable of producing 1,3-butanediol from acetate or acetyl-CoA.

In one embodiment, the non-naturally occurring host cell comprises a modification to either (i) attenuate the activity of an acetaldehyde dehydrogenase enzyme, or (ii) attenuate expression of a gene encoding an acetaldehyde dehydrogenase enzyme. In one embodiment the acetaldehyde dehydrogenase enzyme is a polypeptide having the activity of an enzyme of EC 1.2.1.10. In one embodiment the acetaldehyde dehydrogenase enzyme is endogenous to the non-naturally occurring host cells. In one embodiment, the acetaldehyde dehydrogenase enzyme is exogenous to the non-naturally occurring host cells.

In one embodiment, the non-naturally occurring host cell comprises a modification to either (i) attenuate the activity of an acetolactate decarboxylase enzyme, or (ii) attenuate the expression of a gene encoding an acetolactate decarboxylase enzyme. In one embodiment, the acetolactate decarboxylase enzyme is a polypeptide having the activity of an enzyme of EC 4.1.1.5. In one embodiment the acetolactate decarboxylase enzyme is endogenous to the non-naturally occurring host cells. In one embodiment, the acetolactate decarboxylase enzyme is exogenous to the non-naturally occurring host cells.

In one embodiment, the non-naturally occurring host cell endogenously or exogenously expresses a CoA transferase enzyme. In one embodiment the CoA transferase enzyme is a polypeptide having the activity of an enzyme of EC 2.8.3.-. In one embodiment, the CoA transferase enzyme accepts acetyl-CoA and 3-hydroxybutyryl-CoA. In one embodiment the CoA transferase enzyme is paclJ.

In one embodiment, the non-naturally occurring host cell endogenously or exogenously expresses an acetyl-CoA C-acetyltransferase enzyme. In one embodiment, the acetyl-CoA C-acetyltransferase enzyme is a polypeptide having the activity of an enzyme of EC 2.3.1.9.

In one embodiment, the non-naturally occurring host cell endogenously or exogenously expresses an acetoacetyl-CoA reductase enzyme. In one embodiment, the acetoacetyl-CoA reductase enzyme is a polypeptide having the activity of an enzyme of EC 1.1.1.36.

In one embodiment, the non-naturally occurring host cell endogenously or exogenously expresses a 3-hydroxybutyryl-CoA dehydrogenase enzyme. In one embodiment, the 3-hydroxybutyryl-CoA dehydrogenase enzyme is a polypeptide having the activity of an enzyme of EC 1.1.1.157.

In one embodiment, the non-naturally occurring host cell endogenously or exogenously expresses a carboxylate reductase enzyme. In one embodiment, the carboxylate reductase enzyme is a polypeptide having the activity of an enzyme of EC 1.2.99.6.

In one embodiment, the non-naturally occurring host cell endogenously or exogenously expresses a dehydrogenase enzyme. In one embodiment, the dehydrogenase enzyme is a polypeptide having the activity of an enzyme of EC 1.1.1.-.

In one embodiment, the non-naturally occurring host cell endogenously or exogenously expresses a phosphate acetyltransferase enzyme. In one embodiment, the phosphate acetyltransferase enzyme is a polypeptide having the activity of an enzyme of EC 2.3.1.8.

In one embodiment, the non-naturally occurring host cell endogenously or exogenously expresses an acetate kinase enzyme. In one embodiment, the acetate kinase enzyme is a polypeptide having the activity of an enzyme of EC 2.7.2.1.

In one embodiment, the non-naturally occurring host cell endogenously or exogenously expresses a propionate kinase enzyme. In one embodiment, the propionate kinase enzyme is a polypeptide having the activity of an enzyme of EC 2.7.2.15.

In one embodiment, the non-naturally occurring host cell endogenously or exogenously expresses an alcohol dehydrogenase enzyme. In one embodiment, the alcohol dehydrogenase enzyme is a polypeptide having the activity of an enzyme of EC 1.1.1.1.

In one embodiment, the non-naturally occurring host cell endogenously or exogenously expresses a pyruvate synthase enzyme. In one embodiment, the pyruvate synthase enzyme is a polypeptide having the activity of an enzyme of EC 1.2.7.1.

In one embodiment, the non-naturally occurring host cell endogenously or exogenously expresses an acetolactate synthase enzyme. In one embodiment, the acetolactate synthase enzyme is a polypeptide having the activity of an enzyme of EC 2.2.1.6.

In one embodiment, the non-naturally occurring host cell endogenously or exhogenously expresses an (R,R)-butanediol dehydrogenase enzyme. In one embodiment, the (R,R)-butanediol dehydrogenase enzyme is a polypeptide having the activity of an enzyme of EC 1.1.1.4.

In one embodiment, the non-naturally occurring host cell endogenously or exogenously expresses each of the enzymes set forth in FIG. 1. In one embodiment, the expression of one or more of the enzymes set forth in FIG. 1 has been attenuated in the non-naturally occurring host cells. In one embodiment, the activity of one or more of the enzymes set forth in FIG. 1 has been attenuated in the non-naturally occurring host cells.

In one embodiment, there is a method for producing 1,3-butanediol. In one embodiment the 1,3-butanediol is produced from acetate or acetyl-CoA. In one embodiment, at least one of the steps of the method is performed in a non-naturally occurring host cell. In one embodiment, all of the steps of the method are performed in a non-naturally occurring host cell.

In one embodiment, the method comprises enzymatically converting acetate to acetyl CoA using a CoA transferase enzyme. In one embodiment, the method comprises enzymatically converting 3-hydroxybutyryl-CoA to 3-hydroxybutyrate using a CoA transferase enzyme. In one embodiment, the method comprises using the same CoA transferase enzyme to enzymatically convert acetate to acetyl CoA and to enzymatically convert 3-hydroxybutyryl-CoA to 3-hydroxybutyrate. In one embodiment, the CoA transferase enzyme is a polypeptide having the activity of an enzyme of EC 2.8.3.-. In one embodiment, the CoA transferase enzyme accepts acetyl-CoA and 3-hydroxybutyryl-CoA. In one embodiment, the method is performed in a non-naturally occurring host cell and the acetyl-CoA transferase enzyme is endogenous to the non-naturally occurring host cell. In one embodiment, the method is performed in a non-naturally occurring host cell and the CoA transferase enzyme is exogenous to the non-naturally occurring host cell. In one embodiment, the CoA transferase enzyme is paclJ.

In one embodiment, the method comprises enzymatically converting acetyl CoA to acetoacetyl-CoA using an acetyl-CoA C-acetyltransferase enzyme. In one embodiment, the acetyl-CoA C-acetyltransferase enzyme is a polypeptide having the activity of an enzyme of EC 2.3.1.9. In one embodiment, the method is performed in a non-naturally occurring host cell and the acetyl-CoA C-acetyltransferase enzyme is endogenous to the non-naturally occurring host cell. In one embodiment, the method is performed in a non-naturally occurring host cell and the acetyl-CoA C-acetyltransferase enzyme is exogenous to the non-naturally occurring host cell.

In one embodiment, the method comprises enzymatically converting acetoacetyl-CoA to 3-hydroxybutyryl-CoA using an acetoacetyl-CoA reductase enzyme. In one embodiment, the acetoacetyl-CoA reductase enzyme is a polypeptide having the activity of an enzyme of EC 1.1.1.36. In one embodiment, the method is performed in a non-naturally occurring host cells and the acetoacetyl-CoA reductase enzyme is endogenous to the non-naturally occurring host cell. In one embodiment, the method is performed in a non-naturally occurring host cell and the acetoacetyl-CoA reductase enzyme is exogenous to the non-naturally occurring host cell.

In one embodiment, the method comprises enzymatically converting acetoacetyl-CoA to 3-hydroxybutyryl-CoA using a 3-hydroxybutyryl-CoA dehydrogenase enzyme. In one embodiment, the 3-hydroxybutyryl-CoA dehydrogenase enzyme is an EC 1.1.1.157 enzyme. In one embodiment, the method is performed in a non-naturally occurring host cell and the 3-hydroxybutyryl-CoA dehydrogenase enzyme is endogenous to the non-naturally occurring host cell. In one embodiment, the method is performed in a non-naturally occurring host cell and the 3-hydroxybutyryl-CoA dehydrogenase enzyme is exogenous to the non-naturally occurring host cell.

In one embodiment, the method comprises enzymatically converting 3-hydroxybutyrate to 3-hydroxybutanal using a carboxylate reductase enzyme. In one embodiment, the carboxylate reductase enzyme is a polypeptide having the activity of an enzyme of EC 1.2.99.6. In one embodiment, the method is performed in a non-naturally occurring host cell and the carboxylate reductase enzyme is endogenous to the non-naturally occurring host cell. In one embodiment, the method is performed in a non-naturally occurring host cell and the carboxylate reductase enzyme is exogenous to the non-naturally occurring host cell.

In one embodiment, the method comprises enzymatically converting 3-hydroxybutanal to 1,3-butanediol using a dehydrogenase enzyme. In one embodiment, the dehydrogenase enzyme is a polypeptide having the activity of an enzyme of EC 1.1.1.-. In one embodiment, the method is performed in a non-naturally occurring host cell and the dehydrogenase enzyme is endogenous to the non-naturally occurring host cell. In one embodiment, the method is performed in a non-naturally occurring host cell and the dehydrogenase enzyme is exogenous to the non-naturally occurring host cell.

In one embodiment, the method comprises enzymatically converting acetyl-CoA to acetyl phosphate using a phosphate acetyltransferase enzyme. In one embodiment, the phosphate acetyltransferase enzyme is a polypeptide having the activity of an enzyme of EC 2.3.1.8. In one embodiment, the method is performed in a non-naturally occurring host cell and the phosphate acetyltransferase enzyme is endogenous to the non-naturally occurring host cell. In one embodiment, the method is performed in a non-naturally occurring host cell and the phosphate acetyltransferase enzyme is exogenous to the non-naturally occurring host cell.

In one embodiment, the method comprises enzymatically converting acetyl-phosphate to acetate using an acetate kinase enzyme. In one embodiment, the acetate kinase enzyme is a polypeptide having the activity of an enzyme of EC 2.7.2.1. In one embodiment, the method is performed in a non-naturally occurring host cell and the acetate kinase enzyme is endogenous to the non-naturally occurring host cell. In one embodiment, the method is performed in a non-naturally occurring host cell and the acetate kinase enzyme is exogenous to the non-naturally occurring host cell.

In one embodiment, the method comprises enzymatically converting acetyl-phosphate to acetate using a propionate kinase enzyme. In one embodiment, the propionate kinase enzyme is a polypeptide having the activity of an enzyme of EC 2.7.2.15. In one embodiment, the method is performed in a non-naturally occurring host cell and the propionate kinase enzyme is endogenous to the non-naturally occurring host cell. In one embodiment, the method is performed in a non-naturally occurring host cell and the propionate kinase enzyme is exogenous to the non-naturally occurring host cell.

In one embodiment, the method comprises the enzymatic conversions set forth in FIG. 1. In one embodiment, the method is performed in one or more non-naturally occurring host cells and one or more of the enzymes set forth in FIG. 1 are endogenous to the non-naturally occurring host cells. In one embodiment, the method is performed in one or more non-naturally occurring host cells and one or more of the enzymes set forth in FIG. 1 are exogenous to the non-naturally occurring host cells. In one embodiment, a polypeptide having the activity of the enzyme of EC 1.2.1.10 set forth in FIG. 1 is attenuated. In one embodiment, a polypeptide having the activity of the enzyme of EC 4.1.1.5 depicted in FIG. 1 is attenuated.

In one embodiment, the method consists of the enzymatic conversions set forth in FIG. 2. In one embodiment, the method is performed in one or more non-naturally occurring host cells and one or more of the enzymes set forth in FIG. 2 are endogenous to the non-naturally occurring host cells. In one embodiment, the method is performed in one or more non-naturally occurring host cells and one or more of the enzymes set forth in FIG. 2 are exogenous to the non-naturally occurring host cells.

In one embodiment, at least one of the enzymatic conversions set forth in FIG. 1 or FIG. 2 is performed in a non-naturally occurring host cell.

In one embodiment, at least one of the enzymatic conversions comprises gas fermentation. In one embodiment, the enzymatic conversion is provided by at least one of natural gas, syngas, CO₂/H₂, methanol, ethanol, non-volatile residue, caustic wash from cyclohexane oxidation processes, or waste stream from a chemical or petrochemical industry.

The methods described herein can be performed using isolated enzymes.

The methods described herein can be performed using cell lysates comprising the enzymes.

The methods described herein can be performed in a non-naturally occurring host cell.

In one embodiment, the non-naturally occurring host cell is a prokaryote cell selected from the group consisting of the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens or Pseudomonas putida; from the genus Bacillus such as Bacillus subtillis; or from the genus Rhodococcus such as Rhodococcus equi.

In one embodiment, the non-naturally occurring host cell is a eukaryote cell selected from the group consisting of the genus Aspergillus such as Aspergillus niger, from the genus Saccharomyces such as Saccharomyces cerevisiae; from the genus Pichia such as Pichia pastoris; from the genus Yarrowia such as Yarrowia lipolytica; from the genus Issatchenkia such as Issatchenkia orientalis; from the genus Debaryomyces such as Debaryomyces hansenii; from the genus Arxula such as Arxula adeninivorans; or from the genus Kluyveromyces such as Kluyveromyces lactis.

In one embodiment, there is a method for producing 1,3-BDO by culturing the aforementioned non-naturally occurring host cells under conditions and for a sufficient period of time to produce 1,3-BDO. In one embodiment, the host cells can be subjected to a fermentation strategy entailing anaerobic, micro-aerobic, or aerobic cultivation. A cell retention strategy using a ceramic hollow fiber membrane can be employed to achieve and maintain a high cell density during fermentation.

The principal carbon source fed to the fermentation can derive from a biological or a non-biological feedstock. The biological feedstock can be, or can derive from, monosaccharides, disaccharides, hemicellulose such as levulinic acid and furfural, cellulose, lignocellulose, lignin, triglycerides such as glycerol and fatty acids, agricultural waste or municipal waste. The non-biological feedstock can be, or can derive from, either natural gas, syngas, CO₂/H₂, methanol, ethanol, non-volatile residue (NVR), caustic wash from cyclohexane oxidation processes or other waste stream from either the chemical or petrochemical industries.

According to certain embodiments, the reactions of the pathways described herein can be performed in one or more cell (e.g., host cell) strains (a) naturally expressing one or more relevant enzymes, (b) genetically engineered to express one or more relevant enzymes, or (c) naturally expressing one or more relevant enzymes and genetically engineered to express one or more relevant enzymes. Alternatively, relevant enzymes can be extracted from any of the host cells and used in a purified or semi-purified form. Extracted enzymes can optionally be immobilized to a solid substrate such as the floors and/or walls of appropriate reaction vessels. Moreover, such extracts include lysates (e.g., cell lysates) that can be used as sources of relevant enzymes. In the methods provided, all the steps can be performed in cells (e.g., host cells), all the steps can be performed using extracted enzymes, or some of the steps can be performed in cells and others can be performed using extracted enzymes.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and the drawings, and from the claims. The word “comprising” in the claims may be replaced by “consisting essentially of” or with “consisting of,” according to standard practice in patent law.

### BRIEF DESCRIPTION OF THE DRAWINGS

FIG. 1 is a schematic of an exemplary biochemical pathway leading to 1,3-butanediol.

FIG. 2 is a schematic of an exemplary biochemical pathway leading to 1,3-butanediol.

FIG. 3 is a schematic of an exemplary biochemical pathway leading to 1,3-butanediol.

FIG. 4 depicts the activity of carboxylic acid reductase (CAR) enzymes (D6Z860, A0QWI7, E5XUS9) with 3-hydroxybutyrate and benzoate (as a positive control).

### DETAILED DESCRIPTION

In one aspect, the invention provides enzymes and non-naturally occurring host cells for enhanced 1,3-butanediol production in one or more enzymatic steps. In one aspect, the invention relates to developing and using non-naturally occurring host cells capable of enhanced 1,3-butanediol production by reducing or preventing carbon flux to ethanol and 2,3-butanediol.

In one aspect, there are enzymes and non-naturally occurring host cells for enhanced 1,3-BDO production in one or more enzymatic steps comprising use of one or more of an acetyl-CoA C-acetyltransferase, an acetoacetyl-CoA reductase, a 3-hydroxybutyryl-CoA dehydrogenase, a carboxylate reductase enzyme, a dehydrogenase enzyme, a phosphate acetyltransferase enzyme, an acetate kinase enzyme, and a propionate kinase enzyme; or using non-naturally occurring host cells expressing one or more such enzymes. In one aspect, the non-naturally occurring host cells have attenuated expression or activity of an acetaldehyde dehydrogenase enzyme, an acetolactate decarboxylase enzyme, or both.

Host cells described herein can include pathways that can be manipulated such that 1,3-BDO can be produced. In an endogenous pathway, the host cell naturally expresses all of the enzymes catalyzing the reactions within the pathway. A host cell containing an engineered pathway does not naturally express all of the enzymes catalyzing the reactions within the pathway but has been engineered such that all of the enzymes within the pathway are expressed in the cell.

The term “exogenous” as used herein with reference to a nucleic acid (or a protein) and a host cell refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host cell once in the cell. It is important to note that non-naturally-occurring nucleic acids can contain nucleic acid subsequences or fragments of nucleic acid sequences that are found in nature, provided the nucleic acid as a whole does not exist in nature. For example, a nucleic acid molecule containing a genomic DNA sequence within an expression vector is non-naturally occurring nucleic acid, and thus is exogenous to a host cell once introduced into the cell, since that nucleic acid molecule as a whole (genomic DNA plus vector DNA) does not exist in nature. Thus, any vector, autonomously replicating plasmid, or virus (e.g., retrovirus, adenovirus, or herpes virus) that, as a whole, does not exist in nature is considered to be a non-naturally-occurring nucleic acid. It follows that genomic DNA fragments produced by PCR or restriction endonuclease treatment as well as cDNAs are considered to be non-naturally-occurring nucleic acids since they exist as separate molecules not found in nature. It also follows that any nucleic acid containing a promoter sequence and polypeptide-encoding sequence (e.g., gDNA or genomic DNA) in an arrangement not found in nature is a non-naturally-occurring nucleic acid. A nucleic acid that is naturally-occurring can be exogenous to a particular host cell. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) or a protein and a host cell refers to a nucleic acid or protein that does occur in (and can be obtained from) that particular host cell as it is found in nature. Moreover, a cell “endogenously expressing” a nucleic acid or protein expresses that nucleic acid or protein as does a host of the same particular type as it is found in nature. Moreover, a host cell “endogenously producing” or that “endogenously produces” a nucleic acid, protein, or other compound produces that nucleic acid, protein, or compound as does a host cell of the same particular type as it is found in nature.

For example, depending on the host cell and the compounds produced by the host cell, one or more of the following enzymes may be exogenously or endogenously expressed in the host cell: an acetaldehyde dehydrogenase enzyme, an alcohol dehydrogenase enzyme, a pyruvate synthase enzyme, an acetolactate synthase enzyme, an acetolactate decarboxylase enzyme, an (R,R)-butanediol dehydrogenase enzyme, an acetyl-CoA C-acetyltransferase enzyme, a phosphate acetyltransferase enzyme, an acetate kinase enzyme, a propionate kinase enzyme, an acetoacetyl-CoA reductase enzyme, a 3-hydroxybutyryl-CoA dehydrogenase enzyme, a CoA-transferase, a carboxylate reductase, and a dehydrogenase.

In one embodiment, if the host cell endogenously expresses an acetaldehyde dehydrogenase enzyme, the host cell is modified to attenuate expression or activity of that enzyme. In one embodiment, the acetaldehyde dehydrogenase enzyme is a polypeptide having the activity of an enzyme of EC 1.2.1.10.

In one embodiment, if the host cell endogenously expresses an acetolactate decarboxylate enzyme, the host cell is modified to attenuate expression or activity of that enzyme. In one embodiment, the acetolactate dexarboxylate enzyme is a polypeptide having the activity of an enzyme of EC 4.1.1.5.

In one embodiment, the host cell endogenously expresses a CoA transferase enzyme that accepts both acetate and 3-hydroxybutyryl-CoA. In one embodiment, the host cell is modified to express an exogenous CoA transferase enzyme that accepts both acetate and 3-hydroxybutyryl-CoA. In one embodiment, the CoA transferase enzyme is a polypeptide having the activity of an enzyme of EC 2.8.3.-. In one embodiment the CoA transferase enzyme is paclJ.

In one embodiment, the alcohol dehydrogenase enzyme is endogenous or exogenous. In one embodiment, it is a polypeptide having the activity of an enzyme of EC 1.1.1.1.

In one embodiment, the pyruvate synthase enzyme is endogenous or exogenous. In one embodiment, it is a polypeptide having the activity of an enzyme of EC 1.2.7.1.

In one embodiment, the acetolactate synthase enzyme is endogenous or exogenous. In one embodiment, it is a polypeptide having the activity of an enzyme of EC 2.2.1.6.

In one embodiment, the (R,R)-butanediol dehydrogenase enzyme is endogenous or exogenous. In one embodiment, it is a polypeptide having the activity of an enzyme of EC 1.1.1.4.

In one embodiment, the acetyl-CoA C-acetyltransferase enzyme is endogenous or exogenous. In one embodiment, it is a polypeptide having the activity of an enzyme of EC 2.3.1.9.

In one embodiment, the phosphate acetyltransferase enzyme is endogenous or exogenous. In one embodiment, it is a polypeptide having the activity of an enzyme of EC 2.3.1.8.

In one embodiment, the acetate kinase enzyme is endogenous or exogenous. In one embodiment, it is a polypeptide having the activity of an enzyme of EC 2.7.2.1.

In one embodiment, the propionate kinase enzyme is endogenous or exogenous. In one embodiment, it is a polypeptide having the activity of an enzyme of EC 2.7.2.15.

In one embodiment, the acetoacetyl-CoA reductase enzyme is endogenous or exogenous. In one embodiment, it is a polypeptide having the activity of an enzyme of EC 1.1.1.36.

In one embodiment, the 3-hydroxybutyryl-CoA dehydrogenase enzyme is endogenous or exogenous. In one embodiment, it is a polypeptide having the activity of an enzyme of EC 1.1.1.157.

In one embodiment, the carboxylate reductase is endogenous or exogenous. In one embodiment, it is a polypeptide having the activity of an enzyme of EC 1.2.99.6.

In one embodiment, the dehydrogenase is endogenous or exogenous. In one embodiment, it is a polypeptide having the activity of an enzyme of EC 1.1.1.-.

Within an engineered pathway, the enzymes can be from a single source, i.e., from one species, or can be from multiple sources, i.e., different species. Nucleic acids encoding the enzymes described herein have been identified from various organisms and are readily available in publicly available databases such as GenBank or EMBL.

Any of the enzymes described herein that can be used for 1,3-butanediol production can have at least 70% sequence identity (homology) (e.g., at least 70%, 75%, 80%, 85%, 90%, 95%, 97%, 98%, 98%, 99%, or 100%) to the amino acid sequence of the corresponding wild-type enzyme.

The percent identity (homology) between two amino acid sequences can be determined by any method known to those skilled in the art. In one embodiment, the percent identity (homology) can be determined by aligning the amino acid sequences using the BLAST 2 Sequences (B 12seq) program from the stand-alone version of BLASTZ containing BLASTP version 2.0.14. This standalone version of BLASTZ can be obtained from the U.S. government's National Center for Biotechnology Information web site (www.ncbi.nlm.nih.gov). Instructions explaining how to use the B12seq program can be found in the readme file accompanying BLASTZ. B12seq performs a comparison between two amino acid sequences using the BLASTP algorithm. For example, to compare two amino acid sequences, the options of B12seq are set as follows: -i is set to a file containing the first amino acid sequence to be compared (e.g., C:\seql.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seql.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be used for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

According to certain embodiments, there are (i) functional variants of the enzymes used in the methods of the invention and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes and functional fragments described herein. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the enzymes described herein can also be used in the methods of the invention. The term “functional fragment” as used herein refers to a peptide fragment of a protein that has at least 25% (e.g., at least: 25%; 30%; 40%; 50%; 60%; 70%; 75%; 80%; 85%; 90%; 95%; 98%; 99%; 100%; or even greater than 100%) of the activity of the corresponding mature, full-length, wild-type protein. The functional fragment can generally, but not always, be comprised of a continuous region of the protein, wherein the region has functional activity.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of a recombinant protein (e.g., FLAG, poly histidine (e.g., hexahistidine), hemagluttanin (HA), glutathione-S-transferase (GST), or maltosebinding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Host cells can naturally express none or some (e.g., one or more, two or more, three or more, four or more, five or more, or six or more) of the enzymes of the pathways described herein. Endogenous genes of the host cells also can be disrupted to reduce or prevent the formation of undesirable metabolites, to reduce or prevent the loss of intermediates in the pathway through other enzymes acting on such intermediates, or to reduce or prevent carbon flux to other pathways. Such host cells can be referred to as non-naturally occurring host cells, genetically-modified host cells, recombinant host cells, engineered host cells, or combinations thereof. Thus, as described herein, non-naturally occurring host cells can include modifications, such as genetic modifications, to express and/or attenuate expression of one or more of the enzymes discussed herein.

As used herein, the term “non-naturally occurring,” when used in reference to a microbial organism or microorganism, is intended to mean that the microbial organism has at least one alteration, for example a genetic alteration, not normally found in a naturally occurring strain of the referenced species. Genetic alterations include, for example, modifications including expressible nucleic acids encoding metabolic polypeptides, other nucleic acid additions, nucleic acid deletions, and/or other functional disruption of the microbial organism's genetic material. Such modifications include, for example, coding regions and functional fragments thereof, of heterologous, homologous, or both heterologous and homologous polypeptides for the referenced species. Additional modifications include, for example, non-coding regulatory regions in which the modifications alter expression of a gene or operon. Exemplary metabolic polypeptides include enzymes or proteins within the 1,3-butanediol biosynthetic pathway, the 1,2-butanediol biosynthetic pathway, and/or the ethanol biosynthetic pathway.

A modification that “attenuates” enzyme activity or gene expression in the context of this invention sufficiently reduces or prevents enzyme activity or gene expression so as to reduce or prevent carbon flux in a pathway. In one embodiment, a modification attenuates enzyme activity or gene expression if it inhibits enzyme activity or prevents gene expression. In one embodiment, a modification attenuates enzyme activity or gene expression if it decreases enzyme activity or gene expression. Modifications that attenuate enzyme activity or gene expression include, but are not limited to, point mutations in the enzyme sequence that reduce or abrogate activity, gene knockdowns, gene knockouts, gene silencing, genetic modifications that reduce or prevent transcription of the enzyme, genetic modifications that reduce or prevent translation of the enzyme, deletion of regulatory regions such as promoters or cis binding sites for regulatory factors, truncation of the coding sequence, genetic modifications that upregulate expression or activity of proteins that inhibit the enzyme, genetic modifications that downregulate expression or activity of proteins that activate the enzyme, etc.

In addition, the production of 1,3-BDO can be performed in vitro using isolated enzymes, using a lysate (e.g., a cell lysate) from a host cell as a source of the enzymes, or using a plurality of lysates from different host cells as the source of the enzymes.

In addition, for compounds containing carboxylic acid groups such as but not limited to organic monoacids, hydroxyacids, aminoacids and dicarboxylic acids, these compounds may be formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. The salt can be isolated as is from the system as the salt or converted to the free acid by reducing the pH to below the pKa through addition of acid or treatment with an acidic ion exchange resin.

For compounds containing amine groups such as but not limited to organic amines, aminoacids and diamine, these compounds may be formed or converted to their ionic salt form by addition of an acidic proton to the amine to form the ammonium salt, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid Acceptable inorganic bases include aluminum hydroxide, calcium hydroxide, potassium hydroxide, sodium carbonate, sodium hydroxide, and the like. The salt can be isolated as is from the system as a salt or converted to the free amine by raising the pH to above the pKb through addition of base or treatment with a basic ion exchange resin.

For compounds containing both amine groups and carboxylic acid groups such as but not limited to aminoacids, these compounds may be formed or converted to their ionic salt form by either 1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid Acceptable inorganic bases include aluminum hydroxide, calcium hydroxide, potassium hydroxide, sodium carbonate, sodium hydroxide, and the like or 2) when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. The salt can be isolated as is from the system or converted to the free acid by reducing the pH to below the pKa through addition of acid or treatment with an acidic ion exchange resin.

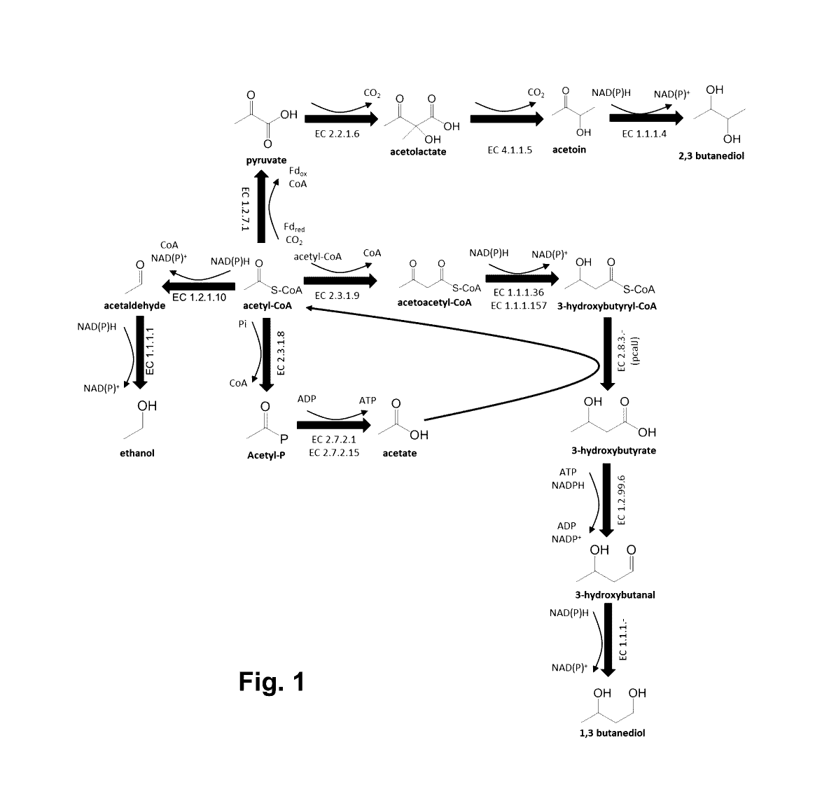

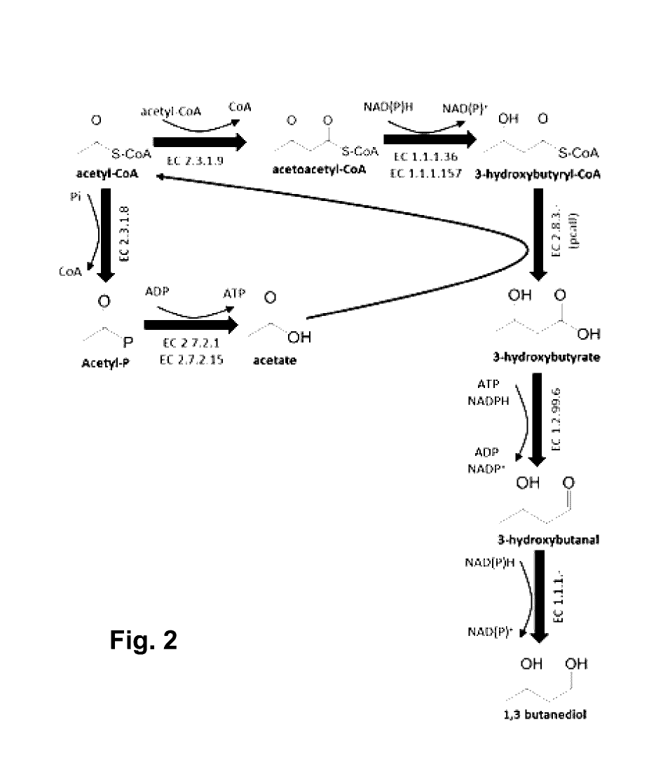

In some embodiments, the enzymes of the pathways described in FIG. 1 and FIG. 2 are the result of enzyme engineering to improve activity or specificity using enzyme structure and wild-type residue diversity to inform rational enzyme design.

In some embodiments, the nucleic acids encoding the enzymes of the pathways described in FIG. 1 or FIG. 2 are introduced into a host cell that is either a prokaryote or eukaryote.

It is understood that when more than one exogenous nucleic acid is included in a non-naturally occurring host cell, that the more than one exogenous nucleic acids can be introduced into the host cell on separate nucleic acid molecules, on polycistronic nucleic acid molecules, or a combination thereof, and still be considered as more than one exogenous nucleic acid. For example, as disclosed herein a non-naturally occurring host cell can be engineered to express two or more exogenous nucleic acids encoding a desired pathway enzyme or protein. In the case where two exogenous nucleic acids encoding a desired activity are introduced into a host cell, it is understood that the two exogenous nucleic acids can be introduced as a single nucleic acid, for example, on a single plasmid or on separate plasmids, or can be integrated into the host chromosome at a single site or multiple sites, and still be considered as two exogenous nucleic acids. Similarly, it is understood that more than two exogenous nucleic acids can be introduced into a host cell in any desired combination, for example, on a single plasmid or on separate plasmids, or can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host cell.

Successfully engineering the pathways of the invention into a host cell involves identifying an appropriate set of enzymes, either cloning their corresponding genes into a production cell or attenuating their expression/activity in the cell, optimizing the stability and expression of the genes, optimizing fermentation conditions, and assaying for product formation following fermentation.

Exogenous nucleic acid sequences involved in a pathway for production of 1,3-butanediol can be introduced stably or transiently into a host cell using techniques well known in the art including, but not limited to, conjugation, electroporation, chemical transformation, transduction, transfection, and ultrasound transformation.

An expression vector or vectors can be constructed to include one or more 1,3-butanediol biosynthetic pathway encoding nucleic acids as exemplified herein operably linked to expression control sequences functional in the host cell. Expression vectors applicable for use in microbial host cells include, for example, plasmids, phage vectors, viral vectors, episomes, and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

Cultivation Strategies

In some embodiments, the host cell is a prokaryote. For example, the prokaryote can be a bacterium from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia such as Delftia acidovorans; from the genus Bacillus such as Bacillus subtillis; from the genus Lactobacillus such as Lactobacillus delbrueckii; or from the genus Lactococcus such as Lactococcus lactis. Such prokaryotes also can be a source of genes to construct non-naturally occurring host cells described herein that are capable of producing 1,3-butanediol.

In some embodiments, the host cell is a eukaryote. For example, the eukaryote can be a filamentous fungus, e.g., one from the genus Aspergillus such as Aspergillus niger. Alternatively, the eukaryote can be a yeast, e.g., one from the genus Saccharomyces such as Saccharomyces cerevisiae; from the genus Pichia such as Pichia pastoris; or from the genus Yarrowia such as Yarrowia lipolytica; from the genus Issatchenkia such as Issatchenkia orientalis; from the genus Debaryomyces such as Debaryomyces hansenii; from the genus Arxula such as Arxula adeninivorans; or from the genus Kluyveromyces such as Kluyveromyces lactis. Such eukaryotes also can be a source of genes to construct non-naturally occurring host cells described herein that are capable of producing 1,3-butanediol.

In some embodiments, 1,3-butanediol is biosynthesized in a non-naturally occurring host cell using a fermentation strategy that can include anaerobic, micro-aerobic or aerobic cultivation of the non-naturally occurring host cell.

In some embodiments, 1,3-butanediol is biosynthesized in a non-naturally occurring host cell using a fermentation strategy that uses an alternate final electron acceptor to oxygen such as nitrate.

In some embodiments, a cell retention strategy using, for example, ceramic hollow fiber membranes can be employed to achieve and maintain a high cell density during either fed batch or continuous fermentation in the synthesis of 1,3-BDO.

In some embodiments, the biological feedstock can be, can include, or can derive from, monosaccharides, disaccharides, lignocellulose, hemicellulose, cellulose, lignin, levulinic acid & formic acid, triglycerides, glycerol, fatty acids, agricultural waste, condensed distillers' solubles, or municipal waste.

The efficient catabolism of crude glycerol stemming from the production biodiesel has been demonstrated in several microorganisms such as Escherichia coli, Cupriavidus necator, Pseudomonas oleavorans, Pseudomonas putida and Yarrowia lipolytica (Lee et al., Appl. Biochem. Biotechnol., 2012, 166, 1801-1813; Yang et al., Biotechnology for Biofuels, 2012, 5:13; Meijnen et al., Appl. Microbial. Biotechnol., 2011, 90, 885-893).

The efficient catabolism of lignocellulosic-derived levulinic acid has been demonstrated in several organisms such as Cupriavidus necator and Pseudomonas putida in the synthesis of 3-hydroxyvalerate via the precursor propanoyl-CoA (Jaremko and Yu, Journal of Biotechnology, 2011, 155, 2011, 293-298; Martin and Prather, Journal of Biotechnology, 2009, 139, 61-67).

The efficient catabolism of lignin-derived aromatic compounds such benzoate analogues has been demonstrated in several microorganisms such as Pseudomonas putida, Cupriavidus necator (Bugg et al., Current Opinion in Biotechnology, 2011, 22, 394-400; Perez-Pantoja et al, FEMS Microbial. Rev., 2008, 32, 736-794).

The efficient utilization of agricultural waste, such as olive mill waste water has been demonstrated in several microorganisms, including Yarrowia lipolytica (Papanikolaou et al., Bioresour. Technol., 2008, 99 (7), 2419-2428).

The efficient utilization of fermentable sugars such as monosaccharides and disaccharides derived from cellulosic, hemicellulosic, cane and beet molasses, cassava, corn and other agricultural sources has been demonstrated for several microorganism such as Escherichia coli, Corynebacterium glutamicum and Lactobacillus delbrueckii and Lactococcus lactis (see, e.g., Hermann et al, Journal of Biotechnology, 2003, 104, 155-172; Wee et al., Food Technol. Biotechnol., 2006, 44 (2), 163-172; Ohashi et al., Journal of Bioscience and Bioengineering, 1999, 87 (5), 647-654).

The efficient utilization of furfural, derived from a variety of agricultural lignocellulosic sources, has been demonstrated for Cupriavidus necator (Li et al., Biodegradation, 2011, 22, 1215-1225).

In some embodiments, the non-biological feedstock can be or can derive from natural gas, syngas, CO₂/H₂, methanol, ethanol, benzoic acid, non-volatile residue (NVR) or a caustic wash waste stream from cyclohexane oxidation processes, or terephthalic acid/isophthalic acid mixture waste streams.

The efficient catabolism of methanol has been demonstrated for the methylotropic yeast Pichia pastoris.

The efficient catabolism of ethanol has been demonstrated for Clostridium kluyveri (Seedorf et al., Proc. Natl. Acad. Sci. USA, 2008, 105 (6) 2128-2133). The efficient catabolism of CO₂ and H₂, which may be derived from natural gas and other chemical and petrochemical sources, has been demonstrated for Cupriavidus necator (Prybylski et al., Energy, Sustainability and Society, 2012, 2:11).

The efficient catabolism of syngas has been demonstrated for numerous microorganisms, such as Clostridium ljungdahlii and Clostridium autoethanogenum (Kopke et al., Applied and Environmental Microbiology, 2011, 77 (15), 5467-5475). Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Although largely H₂ and CO, syngas can also include CO₂ and other gases in smaller quantities. Gasification is generally carried out under a high fuel to oxygen ratio. Numerous gasification processes have been developed, and most designs are based on partial oxidation (to avoid full combustion) of organic materials at high temperatures (500-1500 ° C.) to provide syngas as a 0.5:1-3:1 H₂/CO mixture. Steam is sometimes added to increase the hydrogen content, typically with increased CO₂ production through the water gas shift reaction. Methanol is most commonly produced industrially from the syngas components, CO, and H₂, via catalysis.

The efficient catabolism of the non-volatile residue waste stream from cyclohexane processes has been demonstrated for numerous microorganisms, such as Delftia acidovorans and Cupriavidus necator (Ramsay et al., Applied and Environmental Microbiology, 1986, 52 (1), 152-156).

In some embodiments, substantially pure cultures of non-naturally occurring host cells are provided. As used herein, a “substantially pure culture” of a non-naturally occurring host cell is a culture in which less than about 40% (i.e., less than about 40%; 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the non-naturally occurring host cell, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of non-naturally occurring host cells includes the cells and a growth, storage, or transport medium. Media can be liquid, semi-solid (e.g., gelatinous media), or frozen. The culture includes the cells growing in the liquid or inion the semi-solid medium or being stored or transported in a storage or transport medium, including a frozen storage or transport medium. The cultures are in a culture vessel or storage vessel or substrate (e.g., a culture dish, flask, or tube or a storage vial or tube).

Any of the non-naturally occurring hot cells described herein can be cultured to produce and/or secrete biosynthetic products. For example, 1,3-butanediol producers can be cultured for the biosynthetic production of 1,3-butanediol.

For the production of 1,3-butanediol, the non-naturally occurring host cells may be cultured in a medium with carbon source and other essential nutrients. Anaerobic conditions can be obtained, for example, by first sparging the medium with nitrogen and then sealing the flasks with a septum and crimp-cap. For strains in which growth is not observed anaerobically, microaerobic conditions can be applied by perforating the septum with a small hole for limited aeration. Exemplary anaerobic and aerobic conditions have been described previously and are well-known in the art. Fermentations can be performed in a batch, fed-batch or continuous manner, as disclosed herein.

The pH of the medium may be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

Metabolic Engineering

The present disclosure provides methods involving less than or more than all the steps described for all the above pathways. Such methods can involve, for example, one, two, three, four, five, six, seven, eight, nine, ten, or more of such steps. Where less than all the steps are included in such a method, the first step can be any one of the steps listed. Furthermore, non-naturally occurring host cells described herein can include any combination of the above enzymes such that one or more of the steps, e.g., one, two, three, four, five, six, seven, eight, nine, ten, or more of such steps, can be performed within a host cell.

In addition, where enzymes have been described as accepting CoA-activated substrates, analogous enzyme activities associated with [acp]-bound substrates exist that are not necessarily in the same enzyme class.

Also, where enzymes have been described accepting (R)-enantiomers of substrate, analogous enzyme activities associated with (S)-enantiomer substrates exist that are not necessarily in the same enzyme class.

In addition, where an enzyme is shown to accept a particular co-factor, such as NADPH, or co-substrate, such as acetyl-CoA, many enzymes are promiscuous in terms of accepting a number of different co-factors or co-substrates in catalyzing a particular enzyme activity. Also, where enzymes have high specificity for e.g., a particular co-factor such as NADH, an enzyme with similar or identical activity that has high specificity for the co-factor NADPH may be in a different enzyme class.

In some embodiments, the enzymes in the pathways outlined herein can be the result of enzyme engineering via non-direct or rational enzyme design approaches with aims of improving activity, improving specificity, reducing feedback inhibition, reducing repression, improving enzyme solubility, changing stereo-specificity, or changing co-factor specificity.

In some embodiments, the enzymes in the pathways outlined herein can be gene dosed, i.e., overexpressed, into the resulting non-naturally occurring host cells via episomal or chromosomal integration approaches.

In some embodiments, genome-scale system biology techniques such as Flux Balance Analysis can be utilized to devise genome scale attenuation or knockout strategies for directing carbon flux to 1,3-butanediol.

In some embodiments, fluxomic, metabolomic and transcriptomal data can be utilized to inform or support genome-scale system biology techniques, thereby devising genome scale attenuation or knockout strategies in directing carbon flux to 1,3-butanediol.

In some embodiments, where pathways require excess NADPH co-factor in the synthesis of 1,3-butanediol, a puridine nucleotide transhydrogenase gene such as UdhA can be overexpressed in the host cell (Brigham et al., Advanced Biofuels and Bioproducts, 2012, Chapter 39, 1065-1090).

In some embodiments, where pathways require excess NADPH co-factor in the synthesis of 1,3-butanediol, a glyceraldehyde-3P-dehydrogenase gene such as GapN can be overexpressed in the host cell (Brigham et al., 2012, supra).

In some embodiments, where pathways require excess NADPH co-factor in the synthesis of 1,3-butanediol, a malic enzyme gene such as macA or maeB can be overexpressed in the host cell (Brigham et al., 2012, supra).

In some embodiments, where pathways require excess NADPH co-factor in the synthesis of 1,3-butanediol, a glucose-6-phosphate dehydrogenase gene such as zwf can be overexpressed in the host cell (Lim et al., Journal of Bioscience and Bioengineering, 2002, 93 (6), 543-549).

In some embodiments, where pathways require excess NADPH co-factor in the synthesis of 1,3-butanediol, a fructose 1,6 diphosphatase gene such as fbp can be overexpressed in the host cell (Becker et al., Journal of Biotechnology, 2007, 132, 99-109).

In some embodiments, the efflux of 1,3-butanediol across the cell membrane to the extracellular media can be enhanced or amplified by engineering structural modifications to the cell membrane or increasing any associated transporter activity for 1,3-butanediol.

Producing 1,3-Butanediol Using a Non-Naturally Occurring Host Cell

1,3,BDO can be produced by providing a host cell and culturing the provided cell with a culture medium containing a suitable carbon source as described above. In general, the culture media and/or culture conditions can be such that the cells grow to an adequate density and produce 1,3-butanediol efficiently. For large-scale production processes, any method can be used such as those described elsewhere (see, e.g., Manual of Industrial Microbiology and Biotechnology, 2nd Edition, Editors: A. L. Demain and J. E. Davies, ASM Press; and Principles of Fermentation Technology, P. F. Stanbury and A. Whitaker, Pergamon). Briefly, a large tank (e.g., a 100 gallon, 200 gallon, 500 gallon, or more tank) containing an appropriate culture medium is inoculated with a particular host cell. After inoculation, the host cells are incubated to allow biomass to be produced. Once a desired biomass is reached, the broth containing the cells can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank.

Once transferred, the host cells can be incubated to allow for the production of 1,3-butanediol.

Suitable purification and/or assays to test for the production of 1,3-butanediol can be performed using well known methods. For example, the final product and intermediates, and other organic compounds, can be analyzed by methods such as HPLC (High Performance Liquid Chromatography), UPLC (Ultra Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by, for example, HPLC using, for example, a refractive index detector for glucose and alcohols, and a UV detector for organic acids, or other suitable assay and detection methods well known in the art. The individual enzyme or protein activities from the exogenous DNA sequences can also be assayed using methods well known in the art.

The 1,3-butanediol can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

### EXAMPLE

### Biotransformation of 3-hydroxybutyrate to 3-hydroxybutanal

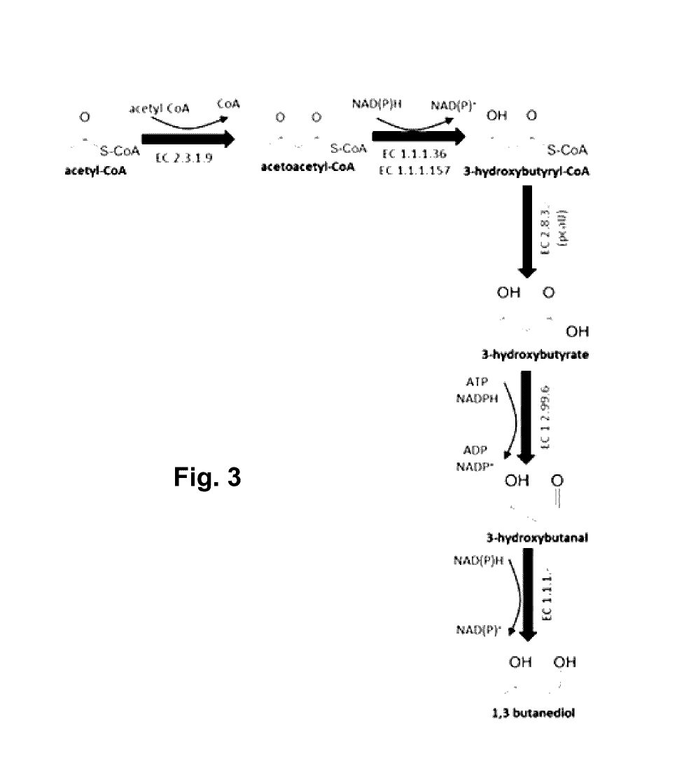

The biotransformation of 3-hydroxybutyrate to 3-hydroxybutanal, depicted in the fourth reaction step of FIG. 3, was measured using Carboxylic acid reductase (CAR) enzymes (D6Z860, A0QWI7, E5XUS9).

CAR enzymes from Segniliparus rugosus (UniProt Access Code: E5XUS9), Seniliparus rotundus (UniProt Access Code: D6Z860), and Mycobacterium smegmatis (UniProt Access Code: A0QWI7) were co-expressed with sfp protein from Bacillus subtilis (UniProt Access Code: P39135) in Escherichia coli BL21(DE3). Expressed enzymes were purified using Histrap columns (GE Healthcare) according to the manufacturer's protocol. Enzyme concentration estimated with Bradford Assay was about 2.5 mg/ml and purity was evaluated with SDS-PAGE. Buffer of the CAR enzymes solutions was exchanged to 50 mM potassium phosphate, pH 6.8, 50 mM NaCl, 5% glycerol.

Assays were performed in disposable transparent NUNC 96-well MTP plates without cover. Typically, a 200 μl reaction mix containing 50 mM HEPES pH 7.5, 10 mM MgCl₂, 1 mM ATP, 0.5 mM NADPH, with or without 2 mM tested substrate (3-hydroxybutyrate or benzoate as a positive control) was prepared. Reactions were started by adding 20 μl CAR enzyme (prepared as described herein) at a final concentration of 0.25 mg/ml. Absorbance at 340 nm, corresponding to the disappearing NADPH, was followed every minute for 1 hour. Negative controls with/without substrate or with/without enzyme were included.

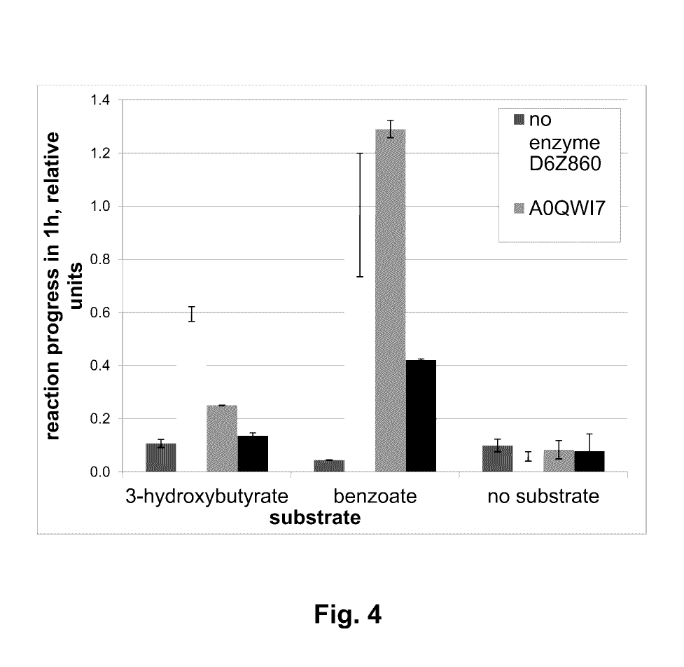

The results are presented in FIG. 4. Benzoate is known as the preferential substrate for CAR enzymes, hence it was used here as a positive control. Results with benzoate confirm that the proteins used in this example are indeed biologically active (FIG. 4). Results with 3-hydroxybutyrate, being clearly above the noise level (no substrate and/or no enzyme), demonstrate that all tested enzymes are able to transform 3-hydroxybutyrate.

### OTHER EMBODIMENTS

It is to be understood that while the invention has been described in conjunction with the detailed description thereof, the foregoing description is intended to illustrate and not limit the scope of the invention. Other aspects, advantages, and modifications are within the scope of the following claims.

1. A non-naturally occurring host cell capable of enhanced 1,3-butanediol production, comprising:
(a) a modification to either:
(i) decrease or inhibit the activity of a polypeptide having the activity of an enzyme of EC 4.1.1.5, or
(ii) decrease or prevent expression of a gene encoding a polypeptide having the activity of an enzyme of EC 4.1.1.5, and
(b) a modification to either:
(i) decrease or inhibit the activity of a polypeptide having the activity of an enzyme of EC 1.2.1.10, or
(ii) decrease or prevent expression of a gene encoding a polypeptide having the activity of an enzyme of EC 1.2.1.10,
wherein, compared to an unmodified host cell, the non-naturally occurring host cell:
(i) gains three reducing equivalents,
(ii) gains three chemical species, wherein said chemical species are capable of transferring the equivalent of one electron in a redox reaction, or
(iii) produces more 1,3-butanediol.

2. The non-naturally occurring host cell of claim 1, wherein the modifications comprise at least one gene knockout.

3. The non-naturally occurring host cell of claim 1, wherein the host comprises one or more exogenous nucleic acids encoding one or more polypeptides having the activity of one or more enzymes chosen from:
(a) an enzyme of EC 2.3.1.9;
(b) an enzyme of EC 1.1.1.36;
(c) an enzyme of EC 1.1.1.157;
(d) an enzyme of EC 2.8.3.-;
(e) an enzyme of EC 1.2.99.6; and
(g) an enzyme of EC 1.1.1.-.

4. The non-naturally occurring host cell of claim 1, wherein the host expresses one or more polypeptides having the activity of one or more enzymes chosen from:
(a) an enzyme of EC 2.3.1.8; and
(b) an enzyme of EC 2.7.2.15.

5. The non-naturally occurring host cell of claim 3, wherein the enzyme of EC 2.8.3.- is pcalJ.

6. The non-naturally occurring host cell of claim 1, wherein the cell is capable of enhanced 1,3-butanediol production from acetate or acetyl-CoA.

7. A non-naturally occurring host cell capable of enhanced 1,3-butanediol production according to the process set forth in FIG. 1, wherein the non-naturally occurring host cell comprises:
(a) a modification to either:
(i) decrease or inhibit the activity of a polypeptide having the activity of the enzyme of EC 4.1.1.5, or
(ii) decrease or prevent expression of a gene encoding a polypeptide having the activity of the enzyme of EC 4.1.1.5, and
(b) a modification to either:
(i) decrease or inhibit the activity of a polypeptide having the activity of the enzyme of EC 1.2.1.10, or
(ii) decrease or prevent expression of a gene encoding a polypeptide having the activity of the enzyme of EC 1.2.1.10,
wherein, compared to an unmodified host cell, the non-naturally occurring host cell:
(i) gains three reducing equivalents,
(ii) gains three chemical species, wherein said chemical species are capable of transferring the equivalent of one electron in a redox reaction, or
(iii) produces more 1,3-butanediol.

8. The non-naturally occurring host cell of claim 7, wherein the modification of (a) comprises a knockout of a gene encoding a polypeptide having the activity of the enzyme of EC 4.1.1.5.

9. The non-naturally occurring host cell of claim 7, wherein the modification of (b) comprises a knockout of a gene encoding a polypeptide having the activity of the enzyme EC 1.2.1.10.

10. The non-naturally occurring host cell of claim 7, wherein the cell is capable of enhanced 1,3-butanediol production from acetate or acetyl-CoA.

11. The non-naturally occurring host cell of claim 1, wherein the cell is a prokaryotic cell.

12. The non-naturally occurring host cell of claim 11, wherein the cell is a prokaryotic cell from the genus Escherichia, Clostridia, Corynebacteria, Cupriavidus, Pseudomonas, Bacillus, or Rhodococcus.

13. (canceled)

14. The non-naturally occurring host cell of claim 12, wherein the cell is a prokaryotic cell from Cupriavidus necator.

15. The non-naturally occurring host cell of claim 1, wherein the cell is a eukaryotic cell.

16. The non-naturally occurring host cell of claim 15, wherein the cell is a eukaryotic cell from the genus Aspergillus, Saccharomyces, Pichia, Yarrowia, Issatchenkia, Debaryomyces, Arxula, or Kluyveromyces.

17. A method for producing 1,3-butanediol in a non-naturally occurring host cell, the method comprising:
enzymatically converting acetate to acetyl CoA using a polypeptide having the activity of an enzyme of EC 2.8.3.-, and
enzymatically converting 3-hydroxybutyryl-CoA to 3-hydroxybutyrate using a polypeptide having the activity of an enzyme of EC 2.8.3.-;
wherein the non-naturally occurring host cell comprises:
(a) a modification to either:
(i) decrease or inhibit the activity of a polypeptide having the activity of an enzyme of EC 4.1.1.5, or
(ii) decrease or prevent expression of a gene encoding a polypeptide having the activity of an enzyme of EC 4.1.1.5, and
(b) a modification to either:
(i) decrease or inhibit the activity of a polypeptide having the activity of an enzyme of EC 1.2.1.10, or
(ii) decrease or prevent expression of a gene encoding a polypeptide having the activity of an enzyme of EC 1.2.1.10,
wherein, compared to an unmodified host cell, the non-naturally occurring host cell:
(i) gains three reducing equivalents,
(ii) gains three chemical species, wherein said chemical species are capable of transferring the equivalent of one electron in a redox reaction, or
(iii) produces more 1,3-butanediol.

18. The method of claim 17, wherein the method further comprises one or more steps chosen from:
enzymatically converting acetyl CoA to acetoacetyl-CoA using a polypeptide having the activity of an enzyme of EC 2.3.1.9;
enzymatically converting acetoacetyl-CoA to 3-hydroxybutyryl-CoA using (i) a polypeptide having the activity of an enzyme of EC 1.1.1.36, or (ii) a polypeptide having the activity of an enzyme of EC 1.1.1.157;
enzymatically converting 3-hydroxybutyryl-CoA to 3-hydroxybutyrate using a polypeptide having the activity of an enzyme of EC 2.8.3.-;
enzymatically converting 3-hydroxybutyrate to 3-hydroxybutanal using a polypeptide having the activity of an enzyme of EC 1.2.99.6; and
enzymatically converting 3-hydroxybutanal to 1,3-butanediol using a polypeptide having the activity of an enzyme of EC 1.1.1.-.

19. The method of claim 17, wherein the method further comprises one or more steps chosen from:
enzymatically converting acetyl-CoA to acetyl phosphate using a polypeptide having the activity of an enzyme of EC 2.3.1.8;
enzymatically converting acetyl-phosphate to acetate using (i) a polypeptide having the activity of an enzyme of EC 2.7.2.1, or (ii) a polypeptide having the activity of an enzyme of EC 2.7.2.15; and
enzymatically converting acetate to acetyl-CoA using a polypeptide having the activity of an enzyme of EC 2.8.3.-.

20. The method of claim 18, wherein the polypeptide having the activity of an enzyme of EC 2.8.3.- is pcalJ.

21. The method of claim 17, wherein the modifications comprise at least one gene knockout.

22. The method of claim 17, wherein the 1,3-butanediol is produced from acetate or acetyl-CoA.

23. A method for producing 1,3-butanediol, comprising performing the enzymatic conversions set forth in FIG. 1 in a non-naturally occurring host cell,
wherein the non-naturally occurring host cell comprises:
(a) a modification to either:
(i) decrease or inhibit the activity of a polypeptide having the activity of the enzyme of EC 4.1.1.5, or
(ii) decrease or prevent expression of a gene encoding a polypeptide having the activity of the enzyme of EC 4.1.1.5, and
(b) a modification to either:
(i) decrease or inhibit the activity of a polypeptide having the activity of the enzyme of EC 1.2.1.10, or
(ii) decrease or prevent expression of a gene encoding a polypeptide having the activity of the enzyme of EC 1.2.1.10,
wherein, compared to an unmodified host cell, the non-naturally occurring host cell:
(i) gains three reducing equivalents,
(ii) gains three chemical species, wherein said chemical species are capable of transferring the equivalent of one electron in a redox reaction, or
(iii) produces more 1,3-butanediol.

24. The method of claim 23, wherein the modification of (a) comprises a knockout of a gene encoding a polypeptide having the activity of the enzyme of EC 4.1.1.5.

25. The method of claim 23, wherein the modification of (b) comprises a knockout of a gene encoding a polypeptide having the activity of the enzyme of EC 1.2.1.10.

26. The method of claim 23, wherein the 1,3-butanediol is produced from acetate or acetyl-CoA.

27. The method of claim 17, wherein the non-naturally occurring host cell is a prokaryotic cell.

28. The method of claim 27, wherein the non-naturally occurring host cell is a prokaryotic cell from the genus Escherichia, Clostridia, Corynebacteria, Cupriavidus, Pseudomonas, Bacillus, or Rhodococcus.

29. (canceled)

30. The method of any of claim 28, wherein the non-naturally occurring host cell is a prokaryotic cell from Cupriavidus necator.

31. The method of any of claim 17, wherein the non-naturally occurring host cell is a eukaryotic cell.

32. The method of any of claim 31, wherein the non-naturally occurring host cell is a eukaryotic cell from the genus Aspergillus, Saccharomyces, Pichia, Yarrowia, Issatchenkia, Debaryomyces, Arxula, or Kluyveromyces.

33. A bio-derived, bio-based or fermentation-derived product comprising:
(a) a composition comprising at least one bio-derived, bio-based or fermentation-derived compound prepared (i) using the non-naturally occurring host cell of any one of claims 1-16, or (ii) according to the method of any one of claims 17-32 or any combination thereof,
(b) a bio-derived, bio-based or fermentation-derived polymer comprising the bio-derived, bio-based or fermentation-derived composition or compound of (a), or any combination thereof,
(c) a bio-derived, bio-based or fermentation-derived resin comprising the bio-derived, bio-based or fermentation-derived compound or bio-derived, bio-based or fermentation-derived composition or compound of (a) or any combination thereof, or the bio-derived, bio-based or fermentation-derived polymer of (b) or any combination thereof,
(d) a molded substance obtained by molding the bio-derived, bio-based or fermentation-derived polymer of (b) or the bio-derived, bio-based or fermentation-derived resin of (c), or any combination thereof,
(e) a bio-derived, bio-based or fermentation-derived formulation comprising the bio-derived, bio-based or fermentation-derived composition or compound of (a), the bio-derived, bio-based or fermentation-derived polymer of (b), the bio-derived, bio-based or fermentation-derived resin of (c), or the bio-derived, bio-based or fermentation-derived molded substance of (d), or any combination thereof, or
(f) a bio-derived, bio-based or fermentation-derived semi-solid or a non-semi-solid stream, comprising the bio-derived, bio-based or fermentation-derived composition or compound of (a), the bio-derived, bio-based or fermentation-derived polymer of (b), the bio-derived, bio-based or fermentation-derived resin of (c), the bio-derived, bio-based, or fermentation-derived resin of (d), or the bio-derived, bio-based or fermentation-derived formulation of (e).

34. A culture medium comprising a bio-derived, bio-based or fermentation-derived compound prepared using the non-naturally occurring host cell of claim 1 wherein the culture medium has been separated from the host cell.

35. A composition comprising the bio-derived, bio-based or fermentation derived product of claim 33 and a compound other than said bio-derived, bio-based or fermentation derived product.

36. The composition of claim 35, wherein said compound other than said bio-derived, bio-based or fermentation derived product is a trace amount of a cellular portion of the host cell or organism.

37. A process for producing the bio-derived, bio-based or fermentation derived polymer of claim 33, comprising chemically reacting the bio-derived, bio-based or fermentation derived product with itself or another compound in a polymer-producing reaction.

38. A process for producing the bio-derived, bio-based or fermentation derived resin of claim 33, comprising chemically reacting said bio-derived, bio-based or fermentation derived product with itself or another compound in a resin producing reaction.

39. The non-naturally occurring host cell of claim 7, wherein the cell is a prokaryotic cell.

40. The non-naturally occurring host cell of claim 39, wherein the cell is a prokaryotic cell from the genus Escherichia, Clostridia, Corynebacteria, Cupriavidus, Pseudomonas, Bacillus, or Rhodococcus.

41. The non-naturally occurring host cell of claim 40, wherein the cell is a prokaryotic cell from Cupriavidus necator.

42. The non-naturally occurring host cell of claim 7, wherein the cell is a eukaryotic cell.

43. The non-naturally occurring host cell of claim 42, wherein the cell is a eukaryotic cell from the genus Aspergillus, Saccharomyces, Pichia, Yarrowia, Issatchenkia, Debaryomyces, Arxula, or Kluyveromyces.

44. The method of claim 23, wherein the non-naturally occurring host cell is a prokaryotic cell.

45. The method of claim 44, wherein the non-naturally occurring host cell is a prokaryotic cell from the genus Escherichia, Clostridia, Corynebacteria, Cupriavidus, Pseudomonas, Bacillus, or Rhodococcus.

46. The method of any of claim 45, wherein the non-naturally occurring host cell is a prokaryotic cell from Cupriavidus necator.

47. The method of any of claim 23, wherein the non-naturally occurring host cell is a eukaryotic cell.

48. The method of any of claim 48, wherein the non-naturally occurring host cell is a eukaryotic cell from the genus Aspergillus, Saccharomyces, Pichia, Yarrowia, Issatchenkia, Debaryomyces, Arxula, or Kluyveromyces.

49. A culture medium comprising a bio-derived, bio-based or fermentation-derived compound prepared using the non-naturally occurring host cell of claim 7, wherein the culture medium has been separated from the host cell.

50. A culture medium comprising a bio-derived, bio-based or fermentation-derived compound prepared according to the method of claims 17, wherein the culture medium has been separated from the host cell.

51. A culture medium comprising a bio-derived, bio-based or fermentation-derived compound prepared according to the method of claims 23, wherein the culture medium has been separated from the host cell.
